(19) United States
(12) Patent Application Publication
(10) Pub. No.: US 2022/0193400 A1
(43) Pub. Date: Jun. 23, 2022

(54) NEURAL INTERFACE

(71) Applicants: Faisal ZAIDI, Brentford (GB); GALVANI BIOELECTRONICS LIMITED, Brentford (GB)

(72) Inventor: Faisal ZAIDI, Spring City, PA (US)

(21) Appl. No.: 17/594,622

(22) PCT Filed: Apr. 24, 2020
(86) PCT No.: PCT/US2020/029863

§ 371 Date: Oct. 25, 2021

Related U.S. Application Data
(60) Provisional application No. 62/838,012, filed on Apr. 24, 2019.

(51) Int. Cl. A61N 1/05; A61N 1/375

(52) U.S. Cl. CPC A61N 1/0558; A61N 1/37518; A61N 1/0556

(57) ABSTRACT

A neuromodulation system is described in which electrodes are mounted within a neural interface device configured to include elastic self-pulsatile or IPG-controlled pneumatic/hydraulic collars in a distal end of the system. One or more electrodes are mounted in each collar. Each collar is connected to an implanted pulse generator (IPG) at the proximal end of the neuromodulation system via a lead body that includes pneumatic/hydraulic lines to the collars and electrical wiring to the electrodes. Gas or fluid pressure supplied to each collar can be controlled by one or more precision step motors inside the IPG. Proximal connectors to the lead body have one or more gas or liquid (biocompatible) reservoirs whose pressure is individually or collectively controlled by the one or more precision motors inside the IPG. The IPG can drive one common or multiple independent precision motors lined around each reservoir to drive one or more collars in the neural interface device.

### RELATED APPLICATION

The present application claims the benefit of U.S. Provisional Application No. 62/838,012 filed Apr. 24, 2019, which is hereby incorporated herein in its entirety by reference.

### TECHNICAL FIELD

The disclosed invention relates generally to the field of electrodes, and more particularly to electrodes employed in the field of neuromodulation. Still more particularly, but not exclusively, the invention relates to neural interfaces for neuromodulation systems.

### BACKGROUND

Typical neuromodulation systems, for example a neural interface for a non-pulsatile nerve targets such as illustrated in FIG. 1, include electrodes permanently embedded in the interior or exterior diameter of a device implanted so that the electrodes of the neural interface are maintained in constant contact with the interior/exterior surface of a target, for example Neuro Vascular Bundle (NVB). The effectiveness of such electrodes can be modulated by turning them on and off. The constant contact between the neural interface and the target such as NVB can lead to the development of tissue reaction (e.g. scar formation) at the contract points between the neural interface and the target tissue such as the NVB, which can impact effectiveness (e.g. electrical effectiveness) over time. Moreover, persistent pressure compression and persistent mechanical irritation of nerves (in a fixed-diameter neural interface design) can contribute to nerve pathology. Such damage can be attributed to (1) excessive compressive pressure damage (mechanical pressure injury from fixed-sized-neural interface—repetitive mechanical compression by a pulsating target causes focal damage); and/or (2) excessive electrode contact duration (persistent electrode-nerve contact triggers a foreign body response leading to local inflammation and epineural fibrosis).

### SUMMARY

The present invention relates to a neuromodulation system comprising a neural interface device including one or more pressure-controlled collars, each of the one or more pressure-controlled collars including one or more electrodes. The neuromodulation system may further comprise an implanted pulse generator (IPG) and a lead body for providing electrical connection between the neural interface device and the IPG. Each electrode may be mounted within a neural interface device configured to include one or more elastic pulsatile pneumatic/hydraulic collars or chambers (hereinafter called a collar) in the distal end of the neuromodulation system, such that the collars are pressure-controlled.

Collar (or platform) is a portion upon which at least a part of one or more electrodes is mountable such that movement of the collar results in a corresponding adjustment in a location of the one or more electrodes. The collar may be moveable by means of pneumatic or hydraulic activation or by means of a spring. The collars may be pressure-controlled by comprising said pneumatic or hydraulic activation means or by comprising a spring and responding to external pressure thus being pressure-controlled. Thus, the collars may be self-adjusting by internal activation means or by responding to external pressure. In embodiments, the collar may be conformable or elastic, whereas in other embodiments the collar may be rigid but movable. In still further embodiments, the collar may be a part of or integrally formed with a mechanical biasing member such as a spring that mechanically biases the electrode toward a target treatment area.

One or more electrodes may be mounted in each collar. In an illustrative embodiment, each collar of the neural interface device may be connected to an implanted pulse generator (referred to as an “IPG”—an implantable device designed to deliver electrical stimulation) at the proximal end of the neuromodulation system via a lead that includes pneumatic/hydraulic lines to the collars and electrical wiring to the electrodes. Gas or fluid pressure supplied to each collar can be controlled by one or more precision motors inside the IPG. Proximal connectors of the lead may have one or more gas or liquid (biocompatible) reservoirs whose pressure is individually or collectively controlled by the one or more precision motors inside the lumen of the IPG. The IPG can drive one common or multiple independent precision motors arranged around each reservoir to drive one or more collars in the neural interface. For example, the motors inside the IPG (or IPG connector) may be precision step-motors.

Peripheral nerves run along with or in close vicinity of blood vessels (artery/veins/lymph vessels), and as a result pulsate with the fluctuating blood pressure inside the vessels at every heartbeat. Splenic nerve (SN) plexus is one such example that run along the splenic artery to the spleen and therefore bear constant pulses of damaging compressive pressure if implanted with fixed-sized cuffs. Nerve bundles sandwiched between a rigid cuff wall and pulsating artery can result in epi/perineurium/axonal thinning, denudation and subsequent degeneration.

Nerve targets may also be nerve bundles that are not part of a vascular structure.

The disclosed neuromodulation system embodiments are designed to safely accommodate such pulsating nerve targets while still providing effective neuromodulation over an extended period. The neural interface device with multiple protruding electrodes sitting over a flexible collar, including a collapsible platform sitting in a trench, avoids the pulsating compressive pressure from the nerves. Compressive pressure intensity (size and stiffness of collars or platform) can be comfortably adjusted via the IPG. The inventive neural interface devices may be extravascular or intravascular. An implanted neural interface device may initially be in a deployed configuration, with the electrodes (protruded; collars inflated) contacting the nerve targets until such time that fibrotic tissue surrounds the cuff cements the cuff in place. Once stabilized, the electrodes may be withdrawn (parked in a pressure-less sleeping state) by simply deflating the collars and inflating (deploying collars; electrodes protruded) only when the stimulation is desired.

In various embodiments, the neuromodulation systems described herein can include various deployable portions. In some embodiments, an entire arm or cuff may be conformably adjusted, while in other embodiments individual electrodes or electrode plates may be adjustable. Self-adjusting portions may be used to reduce an amount of overall contact area with portions of a patient that are sensitive to abrasion- or pressure-induced damage, such as nerve bundles, neurovascular bundles or blood vessels. In some embodiments only electrode contacts are in physical contact with a target area, rather than than cuff arms or other components, which significantly reduces physical contact with the target and either reduces or even eliminates potential tissue irritation sources.

In embodiments, at least some parts of the cuff can be in contact with the target, while in others the cuff may be in contact with the target while one or more electrodes are retracted. The size of the cuff (or an arm of the cuff) can be larger than the target so that there can be distancing between the cuff and the target, to avoid physical contact other than at the electrode, in embodiments. In embodiments where the cuff is larger than the target and the electrode position is variable, the cuff can be fit-for-all size and design. Additional features of the inventive system are described below.

### BRIEF DESCRIPTION OF THE DRAWINGS

FIG. 1 is a cross-sectional view of an example implantable fixed-diameter cuff encircling the nerve bundle.

FIG. 2A is an illustration of an extravascular design for a neural interface in accordance with the present invention with self-adjusting electrodes.

FIG. 2B is a partial view of an extravascular design for a neural interface in accordance with the present invention with self-adjusting electrodes.

FIG. 2C is a view of the design of FIG. 2B with the arms depicted as translucent to detail internal active components.

FIG. 3 depicts an intravascular design for a neural interface device in accordance with the present invention.

FIG. 4A a zoomed in view of a self-adjusting electrode for both extravascular and intravascular embodiments.

FIG. 4B is a view of the electrode of FIG. 4A with the collar depicted as translucent to detail internal components.

FIG. 4C is a view of self-adjusting electrode without the collar depicting electrode motions and detail internal components.

FIG. 5 depicts an IPG header design with proximal connector inserted into the header of the smart IPG.

FIG. 6 depicts an IPG design including pneumatic/hydraulic controls built within the IPG.

FIG. 7 depicts a lead body configuration.

FIG. 8 depicts an example of a self-sizing collar and electrode layout.

FIG. 9 depicts an example of a pulsatile collar and electrode platform in a trench layout.

### DETAILED DESCRIPTION OF ILLUSTRATIVE EMBODIMENTS

The disclosure relates to systems and devices for stimulating a nerve intra- or extra-vascularly, in the case of NVB implantations, or other nerve targets through use of an implantable device that includes one or more electrodes positioned at the distal end of a lead or within a cuff or similar device implanted within a vein/artery or around the outside of a vein/artery or nerve such that the electrodes may be in contact with surface tissue during stimulation. The implantable device may include a neural interface with one or more electrodes that are self-sizing (and/or electrically controlled). Stimulation of the nerve may be defined by the delivery of electricity (e.g., electrical pulses) to a neuron, a nerve cell, a nerve bundle, or other target location of the nervous system that excites the neuron, nerve cell, nerve bundle, or other target location.

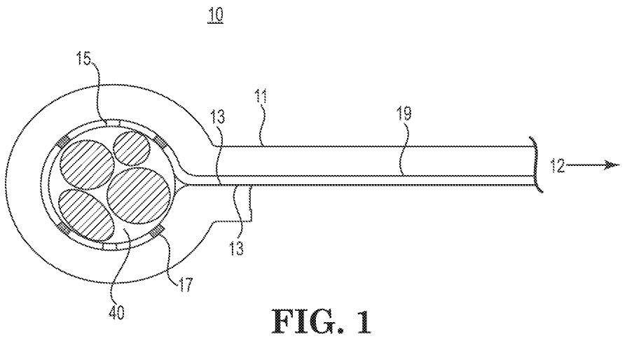

FIG. 1 depicts an example system of an implantable device 10 formed of a flexible, biocompatible material 11, such as a soft polymer substrate, that may be used to stimulate a nerve using energy generated from a voltage induced in the implantable device by a source 12 (not otherwise shown in FIG. 1). As shown, the implantable device 10 is an extra-vascular device in the form of a cuff that is wrapped around a nerve or blood vessel 40 and joined together at points 13. The implantable device 10 is merely an illustrative example and may come in many different shapes, configurations, sizes, etc.

The implantable device 10 may include one or more sensors 15 and electrode arrays 17, each comprising one or more sets of electrodes. Each electrode array 17 may be configured to emit electrical fields to stimulate nerve bundles 40 proximate to the implantable device 10. Each set of electrodes within electrode array 17 may include one or more individual electrodes for this purpose. In some embodiments, each electrode may reflect a micro-electrode designed for maximum charge injection for stimulating a nerve.

Each electrode within an electrode array 17 (or a subset of such electrodes, etc.) may be coupled to one another via a high-density, flexible interconnection or lead 19 made substantially out of conductive material. In some embodiments, for example, interconnection or lead 19 may be comprised substantially (e.g., 90 or 95 percent by weight) out of metals such as platinum or titanium. Other metals, such as gold, may also be used for interconnection or lead 19, such as for electrical wiring within the lead 19. As depicted in FIG. 1, the electrodes within electrode array 17 may be connected via the electrical wiring within lead 19 in series and/or parallel to provide multiple channels for increased selectivity of the parameters of the emitted electric field (e.g., magnitude, direction, location, etc.).

The electrode array 17, its component set of electrodes, and/or individual electrodes within the array may be coupled to one or more other components of implantable device 10 for conducting processes consistent with the disclosed embodiments. These couplings may occur through lead 19, couplings made from the same or similar materials, or other couplings electrically linking the coupled components. In some embodiments, for instance, the electrode array 17 and/or its component sets of electrodes or individual electrodes may be coupled via the wiring in lead 19 to a control circuit, a battery, capacitive storage and/or other chargeable storage elements to emit an electric field to stimulate a proximate nerve in response to a control signal received from the control circuit.

The one or more sensors 15 may also be electrodes or arrays of electrodes that measure a physical or temporal parameter associated with implantable device 10 and/or its surroundings. For example, in one embodiment, the set of sensors 15 may include sensors for measuring the electrical potential between two points. In addition, the set of sensors 15 may include other sensors for measuring other characteristics such as pressure, temperature, time, resistance, conductance, electrical/magnetic flux, and so forth. Each sensor in the set of sensors 15 may be coupled to any other component of implantable device 10, such as the control circuit, electrode arrays 17, energy sources, etc.

Each of the components of implantable device 10 may be formed within or affixed into the soft polymer substrate 11 so that the substrate supports the formed or affixed components. In certain embodiments, the substrate 11 may comprise a single piece of flexible polymer material, such as silicone, to facilitate implantation into a patient and manipulation therein. In some embodiments, the substrate 11 may comprise a plurality of layers of material with various components, such as electrode arrays 17, sensors 15 and lead 19 positioned between the layers.

Neural Interface Device

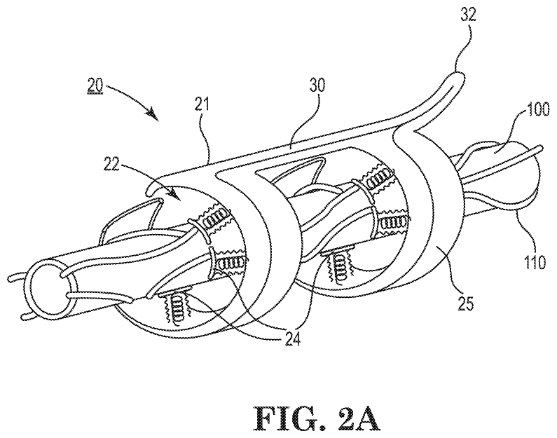

We will now describe the inventive neural interface (NI) devices for controlling a neural interface (NI) to target nerve tissue. The inventive neural interface devices can control the NI size, pressure, and contact time, i.e., contact (nerve-electrode) duration. The neural interface device designs may be intravascular or extravascular, as further shown herein. Extravascular designs include various cuff designs, including helix or serpentine designs. Applicant's own prior designs also include Venus Flytrap type among others (as shown and/or described in PCT/GB2018/052076, published as WO 2019/020985). Intravascular designs include simple lead-like or catheter-like designs with the collar-controlled electrodes configured at the distal end. With respect to size control, in a traditional neural interface device design, such as that shown in FIG. 1, the size of the NI never changes once implanted. While electrodes may be turned on or off, the size set of electrodes for the NI remains the same at all times. The inventive neural interface devices make it possible to change the effective size of the NI (as dictated by the anatomy of the target) in real time. Target NVB anatomy (size) changes with time as the age progress. In a cuff, for example, with multiple electrodes physically controlled by different collars, the size of the NI may be based on any combination of available electrodes determined by the size and the number of collars. For example, as shown in FIG. 2A, a cuff embodiment includes a total of six electrodes each mounted on a separately controllable collar. The NI can also be a single electrode, achieved by activating just one collar so the electrode of that collar is in contact with the target tissue, or any other combination of the electrodes by activating different collars (in some embodiments, the arms of the cuff do not directly contact the NVB).

With respect to pressure, in a traditional neural interface device design, such as that shown in FIG. 1, the amount of pressure asserted by each electrode on the target tissue does not change once the cuff has been implanted. The interface compressive pressure increases with the increase in the NVB target size/age. The inventive designs make it possible to regulate the amount of pressure applied by each electrode mounted on a controllable collar.

Air or fluid pressure (inside the collar) is regulated such that the contact pressure applied is just enough to enable the electrode to protrude and make a physical contact with the target NVB. Further pressure may be regulated so that the contact pressure is light, heavy or anywhere in-between.

With respect to positional duration, traditional neural interface device designs do not regulate target tissue contact or contact time, i.e., once the device is in place, it remains in place during the duration of its implantation with the electrodes in constant contact with the target tissue the entire life. While the electrodes may be turned on or off during neuromodulation, the electrodes remain in physical contact even when off and not in use (e.g. not stimulating). The inventive designs make it possible to only have the electrodes in contact with the target tissue when the electrodes are delivering stimulation current or in use for other activities such as nerve recordings or impedance recordings etc. When the electrodes are off or not in use, the collars may be retracted so that the electrodes are not in physical contact with the target NVB. This design may significantly reduce persistent/chronic mechanical irritation of the target and may prevent scar tissue (or fibrous tissue) development or tissue reaction, for example foreign body reaction.

Neuromodulation System

In an embodiment, the neuromodulation system may include the following components:

1. Neural Interface: As described above, the neural interface device located at the distal end of the system may include intravascular and extravascular designs. The intravascular designs may be catheter-like, designed to be positioned in the middle of the lumen of the blood vessel in which it is implanted, but not otherwise touching the interior diameter of the lumen (e.g. vessel wall) when the collars of the electrodes are not activated or otherwise deployed (protruded/extended). The extravascular designs may include cuffs of various configurations, including helix or serpentine designs, Venus Flytrap-type designs, etc. All of the neural interface device designs can be configured to include one or more pneumatic and/or hydraulic collars or platforms for the electrodes as further enumerated below.

2. Maneuverable or Self-Pulsatile Pneumatic Collar: Electrodes may be mounted on the target tissue facing ends of one or more elastic self-pulsatile pneumatic collars of the neural interface device. In another design each collar may be independently connected to pneumatic lines that provide pressurized biocompatible gas to each collar. Gas pressure inside each collar is controlled by the IPG at the proximal end of the neuromodulation system. In an embodiment, the IPG includes a header that includes one or more precision motors, such as step or stepper motors, as well as circuitry and other components for controlling the stepper motors. As further described herein, the header may also include one or more corresponding reservoirs for holding the gas or liquid. The invention, however, is not limited to the motor, the reservoirs, or other components being located in the header; in embodiments, the motors, reservoirs and other components may be located elsewhere.

To activate a collar and cause the electrode(s) of the collar to make contact with the target tissue, the stepper motors may be activated to apply pressure to the gas reservoir embedded inside the proximal connector of the lead, which would in turn increase pressure within the closed system of the pneumatic lines and cause the collars to inflate (protrude/extend). A change in inductance or capacitance across the electrode (which may be sensed and communicated via electrical leads) would identify to the neuromodulation system that an electrode is in contact with target tissue, at which time the amount of pressure could be further controlled to regulate how firmly the electrode is pressed against the target tissue and to identify when the electrode can be turned on for neuromodulation. Depending on the configuration of the neural interface device and the configuration of the IPG, collars may be individually or collectively controlled to activate at the same or different times, thereby also controlling the size of the neural interface and the positional duration (e.g. timing and duration) of neuromodulation applied to the target tissue. For example, there may be multiple gas reservoirs in the proximal connector of the lead, each of which is pressurized by a different stepper motor or set of opposing stepper motors and each of which has a separate pneumatic line connected to a separate collar. Such an embodiment would enable individual control of each collar and corresponding electrode or electrode array of the collar. Alternatively, multiple collars may share a common pneumatic line and common reservoir and be controlled by the same stepper motor or set of stepper motors.

3. Maneuverable or Self-Pulsatile Hydraulic Collar: The elastic pulsatile hydraulic collars may be configured in the same manner as the elastic pulsatile pneumatic collars, only using a biocompatible fluid in place of gas. Fluid pressure inside each collar may be precisely controlled by stepper motors of the IPG, which may be located inside the IPG header.

4. Maneuverable or Self-Pulsatile Hydraulic/Pneumatic Platform: The neural interface device may include an elongated collar in the shape of a flat platform that holds some or all of the electrodes of the neural interface device and a fitting trench or trough. There may be one or more platforms per neural interface device. The platform may be self-pulsatile or change its position in and out of the trench. The platform may be controlled through pneumatic/hydraulic lines connected to an IPG including at least one gas/fluid reservoir per platform and one or more stepper motors for applying pressure to the reservoirs based on control signals from the IPG's electronic circuitry. Each neural interface device may include a trench or trough in which the platform rests (e.g. withdrawn in the trench/trough) when deactivated (retruded). When deactivated, the electrodes of the platform would not be in contact with the target tissue. When activated by sufficient pressure applied by the stepper motor(s) to the reservoir(s), the platform would rise out of the trench or trough and make contact with the target tissue along the length of the platform whenever desired.

5. IPG: The IPG may be an implantable device that includes a hermitically sealed can for storing electrical components and circuitry. The can may include a battery, or the battery may be external to the can and separately hermitically sealed. The IPG may further include a lid that closes a top of the can and provides feedthroughs for wiring between the can and a hermitically sealed header typically mounted on the lid. The header may include one or more connectors for connecting to the lead to the neural interface device as well as antennas or coils for enabling the IPG to communicate with other devices outside of the body in which the IPG is implanted and to wireless recharge the battery. The header may also include one or more stepper motors for precisely applying pressure to the one or more reservoirs although the reservoirs and stepper motors could be located in the can. The header may also include one or more reservoirs for holding gas or liquid for controlling pressure in pneumatic or hydraulic lines to the collars.

The reservoirs and stepper motors could also be located external to the IPG, such as in a separately implantable intermediate device that provides an interconnection between the IPG and the lead to the neural interface device. Also, although embodiments described herein illustrate either pneumatic or hydraulic systems, the neuromodulation system could include both pneumatic and hydraulic system, which would require separate reservoirs and lines for holding gas and liquid. For example, there could be separate intermediate devices, one for gas and one for liquid, or one device that combined both, or both could be combined in the same IPG.

The electronic circuitry in the IPG may be configured to control the stepper motors, regardless of where the stepper motors may be located, to deliver controlled electrical pulses to the electrodes, and to receive signals for sensing electrodes that sense capacitance, inductance, action potential and other signals necessary to control the neural interface device.

6. Lead: The lead may be biocompatible conduit for multiple electrical wires conducting electrical current to the electrodes and multiple lines (or tubes) conducting pressurized fluid and/or and gas to the neural interface. The lead may run from the IPG to the neural interface or from an intermediate device to the neural interface, which would also require electrical leads between the intermediate device and the IPG for providing power and electrical control signals to the intermediate device. The lead body may be undulated (i.e., a zig-zag configuration), flexible, stretchable, patterned, with stain relief configurations, inbuilt suture-sleeves and/or patterns. Each corner (turn, or angle) of the lead may be stabilized with rigid molds (e.g. plastic molds) to retain desired shapes. These molds may prevent lead body migration during high pneumatic/hydraulic pressure pulses.

Extravascular/Intravascular Designs

FIGS. 2A, 2B and 2C are illustrations of designs for an extravascular neural interface device 20 in accordance with one embodiment, as shown in FIG. 2A, and a second embodiment, shown in FIGS. 2B and 2C. In FIG. 2A, the neural interface device 20 is in the form of a cuff 21 that includes a plurality of collars 22 arranged around arms 25 of the cuff 21. Each of the collars 22 includes an electrode or electrode array 24, generally configured as shown in relation to an artery 100 and nerves 110. FIGS. 2B and 2C show the neural interface device 20 without the artery and nerves illustrated in FIG. 2A. FIG. 2C shows the same portion of the neural interface device 20 illustrated in FIG. 2B but the arms are translucent, so the active components are more clearly illustrated. In FIGS. 2B and 2C, the cuff 23 includes arms 25, each including a plurality of collars 22, each having an electrode or electrode array 24, and includes an additional positional arm 27 that does not include any active elements. In FIGS. 2A, 2B and 2C, one set of electrical wires and gas/liquid lines 30 (or individual wires/lines) run from lead 32 to the collars 22 such that the same (or different) electrical pulses are provided to each of the electrodes 24 and the same (or different) amount of pressurized gas or liquid is provided to each of the collars 22.

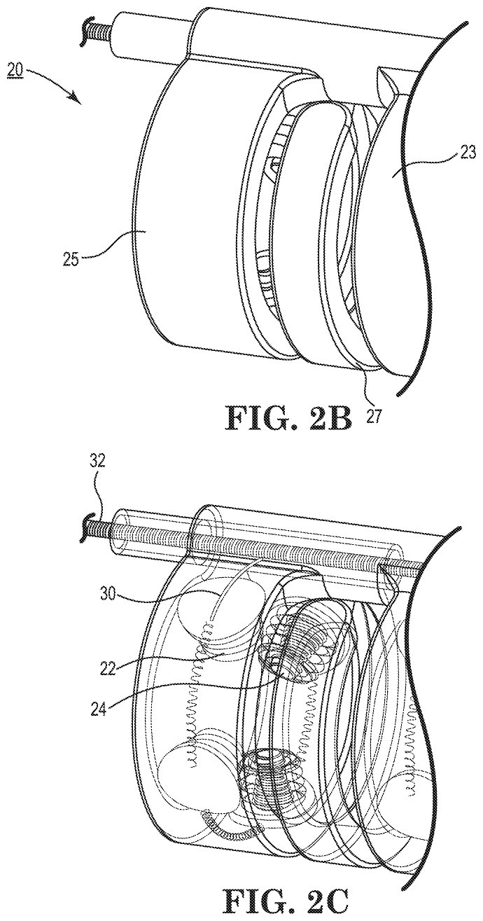

FIG. 3 depicts an intravascular (catheter like) design for the neural interface device 90. As shown, the neural interface device 90 is placed within a lumen 120 of the artery 100 and nerves 110. The neural interface device 90 may be shaped much like the lead 32 of FIG. 2A, which includes a plurality of electrical wires and gas/liquid control lines/tubes 26. The device 90 includes a number of collars 22 that extend from the catheter-like body, each of which includes an electrode or electrode array 24. The collars 22 may include a common gas/liquid line 26 that is shared between each of the collars 22, such that all collars are activated and deactivated at the same time, or each collar 22 may have a separate control line 26 and be self-adjusting. Likewise, each of the electrodes 24 may share a common electrical wire and be activated and deactivated at the same time, or each electrode 24 may have a separate electrical wire for individual control. The lead 32 would exit the lumen 120 in a known manner at a different location in the body in which the device 90 was implanted.

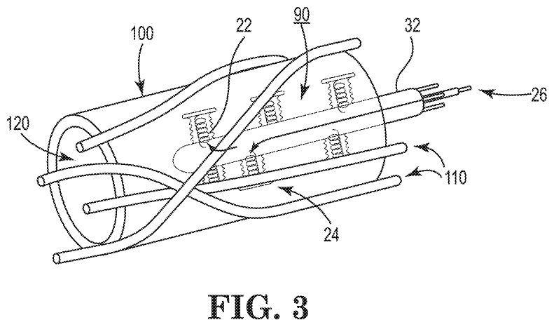

FIG. 4A is a zoomed in illustration of the self-adjusting electrode 24 of the above designs or embodiments. The electrode 24 is suitable for both extravascular and intravascular implementations. FIG. 4B illustrates the electrode 24 with the elastic, self-pulsatile pneumatic collar 22 as translucent to better illustrate the internal components. The externally visible components include a base 34 and an electrode contact 35. The internal components include a collar 22 of electrode plate 36 of the electrode contact 35, and a flexible lead 37 around a support 38. The flexible lead 37 provides an electrical connection to the electrode contact 35. The support 38 positions and supports the lower portion of the flexible lead 37. Gas or liquid may enter (to activate) and exit (to deactivate) the collar 22 in or near the support 38.

Each of the above designs or embodiments share some common attributes. Each embodiment allows the pressure, position and duration of the NI to be controlled. Pressure control can be adjusted by either a pneumatic collar or a hydraulic collar and positional (ON or OFF) application and durational (ON/OFF time) application can be controlled by activating and deactivating the collars. When the collar is deactivated (deflated), the electrodes are no longer in physical contact with the target tissue (e.g., the electrodes may be retruded or collapsed). Size control can be achieved in embodiments that have separately controllable collars, which may enable some collars to be activated (protruded/inflated) and others to be deactivated (retracted). An advantage to pressure control is that it may relieve pressure from the nerves when the electrodes are not in use. In traditional designs, such as that shown in FIG. 1, the nerves are permanently sandwiched between the arterial walls and the cuff (and the cuff electrodes) at all times. This may place the nerves under pressure. An advantage to positional duration is that it may reduce the duration of the electrode-nerve contact. In other words, electrodes are deployed only when needed, thereby limiting foreign body reaction/response. An advantage to size control is that it may allow for different configurations of the electrodes fitting different sized NVB anatomies that would otherwise be impossible. In other words, some collars may be fully activated when other collars are only partially, thereby making it possible to regulate the circumferential size (diameter) of the neural interface in ways not permitted by traditional devices. An advantage to the overall design of the neural interface devices is that only the metal electrodes touch the NVB reversibly adjust to the size of the NVB target and contact the target tissue only when the collars are inflated (i.e., when electrodes are providing stimulation). Thus, in some embodiments the cuff arms or cuff arm material does not touch the target, such as the NVB. In traditional designs, the neural interface (both cuff material and electrode metal) is always in contact with NVB, other portions or material of the neural interface device (such as arms) are also in contact with the target tissue, such that the neural interface cannot adjust to the pulsating/changing size of the NVB target. This can contribute to a foreign body reaction.

In some instances, pressure injury may result in nerve pathology caused by repetitive mechanical compression to the nerve by the pulsating arterial target. Accordingly, embodiments are envisioned in which cuff size is not fixed, which may be appropriate in some instances. For instance, variable-sized cuffs may be used along a target such as nerves (e.g. peripheral nerves) that run along with or in close vicinity of blood or lymph vessels. Some of the nerve targets for effective application of the devices described herein are pulsating NVBs, and the pulsation of the NVBs can benefit from a forgiving or conformable fit that maintains desired levels of contact without causing excess compressive force on the NVB as the arteries or other body fluid vessels may naturally pulsate with the heartbeat. This conformable embodiment reduces thinning or loss of the tissue layers or degeneration of the nerve or nerve bundle's function.

In embodiments, self-adjusting electrode 24 may use a spring-mounted electrode rather than the pressure-controlled collar described above. FIG. 4C demonstrates electrode 24 mounted on an elastic spring 39 in both a protruded/deployed and a compressed/non-deployed configuration. The elastic spring 39 may be an independent component, or it may be integrated with other components of the neural interface. The springs may be constant-pressure/force spring. Flexible lead 37 may for instance be a self-adjusting elastic (constant-pressure/force) spring capable of free pulsation with arterial blood pressure. In embodiments, flexible lead 37 may be unchanged from the above description and support 38 may comprise a self-adjusting elastic spring capable of free pulsation. Embodiments using self-adjusting elastic springs can enable non-restricted low pressured free pulsation to the NVB which may help relieve pressure on nerves which may lie between the electrode cuff and an arterial wall. The compressive intensity of the collar, determined according to the stiffness of the spring, may be self-adjusted or fixed. In addition to spring-mounted or pressure-controlled versions of the collar as shown in FIGS. 4A-4C, other mechanical biasing members may be used in other embodiments.

IPG Designs

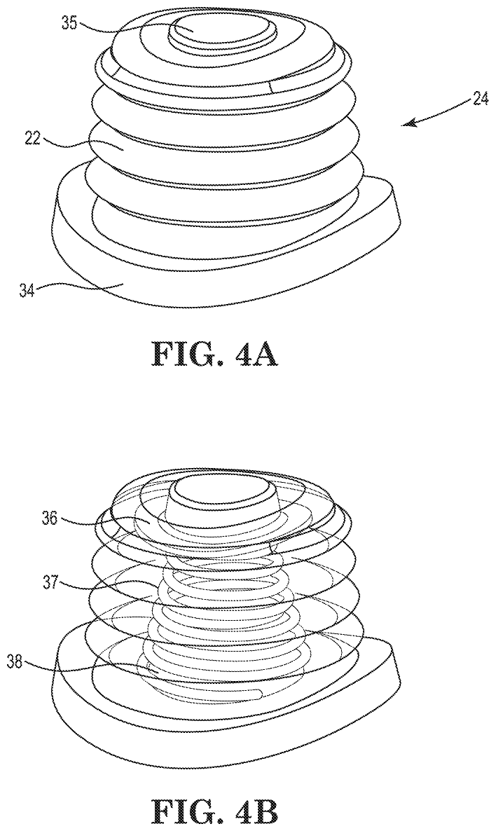

FIG. 5 depicts a design for an IPG 40 in which reservoirs and stepper motors are incorporated into the header 48 of a can body 49. IPG headers have female connectors that have a series of contacts that correspond to a series of contacts on a male connector. FIG. 5 provides a simplified illustration of this concept in which the proximal end of the NI lead splits into two proximal inline connectors. Proximal connector 41 has a series of inline electrical connectors that provide all of the contacts to the current line 42 of the lead 32. Likewise, proximal connector 45 has a series of inline electrical connectors that provide all of the reservoirs to the pressure line 44 of the lead 32. The illustrated design provides a total of 8 connections for a corresponding number of electrical wires or pressure lines in the lead 32 and a corresponding number of electrodes in a connected neural interface device (not shown). FIG. 5 also provides a simplified illustration of a series of stepper motors 46 each positioned adjacent to a reservoirs in the proximal connector. Each of the 8 reservoirs are in turn connected to one line for the pressure (pneumatic or hydraulic) control line 44.

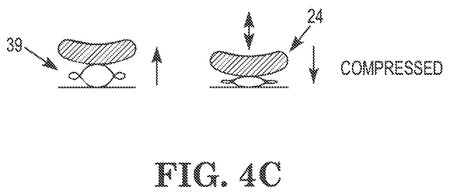

FIG. 6 depicts a further simplified illustration of an IPG 50 that includes one or more reservoirs 56 that are located inside the IPG header. In this embodiment, stepper motors 58 inside the IPG header are positioned against each reservoir 56 filled with either gas or liquid. Only one set of opposing stepper motors 58 are illustrated relative to one reservoir 56 to simplify the illustration. Each reservoir is proximally connected to a hydraulic or pneumatic line 59 that is distally connected to a collar 52 and 54. Electrical wires may be run contiguous with the line 59 to connect stimulation controllers within the IPG with the electrodes 55 of each collar 52 and 54. The electrodes 55 may be deployed by controlling the length of the collars 52 and 54, which are made of a biocompatible material that is elastic in nature. The collars 52 and 54 have a metal spring supporting the electrode plate to the metal base. Electrode contact time and pressure may be controlled by application of the stepper motors 58 on the pneumatic/hydraulic reservoirs 56 inside the IPG. In FIG. 6, a first collar 52 is shown retracted (or deactivated) away from the target tissue (so as to not make contact with the target tissue) since its pressure is reduced in its corresponding hydraulic or pneumatic line resulting in a deflated or deactivated condition 52a. When the pressure in the corresponding hydraulic or pneumatic line is reduced, the compression spring automatically withdraws and pulls the electrode away from the nerve bringing it closer to the base. On the other hand, a second collar 54 is illustrated as deployed or protruded or pushed out toward the target tissue (to make contact with the target tissue) as pressure in the corresponding hydraulic or pneumatic line results in an inflated or activated condition 54a.

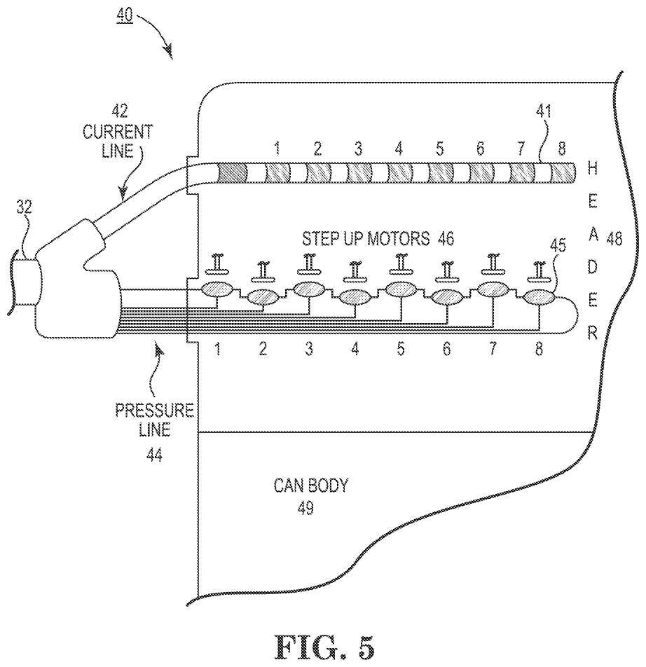

FIG. 7 depicts a lead body configuration 60 including multiple hydraulic/pneumatic lines (conduits/tubes) 62 may be arranged around the central chamber of electrical wires 66 and then covered with a biocompatible material to hermetically seal the wires 66 and lines 62 from body fluid contact. Each electrode and each collar may be independently controlled via its own unique electrical and pneumatic/hydraulic lines, respectively.

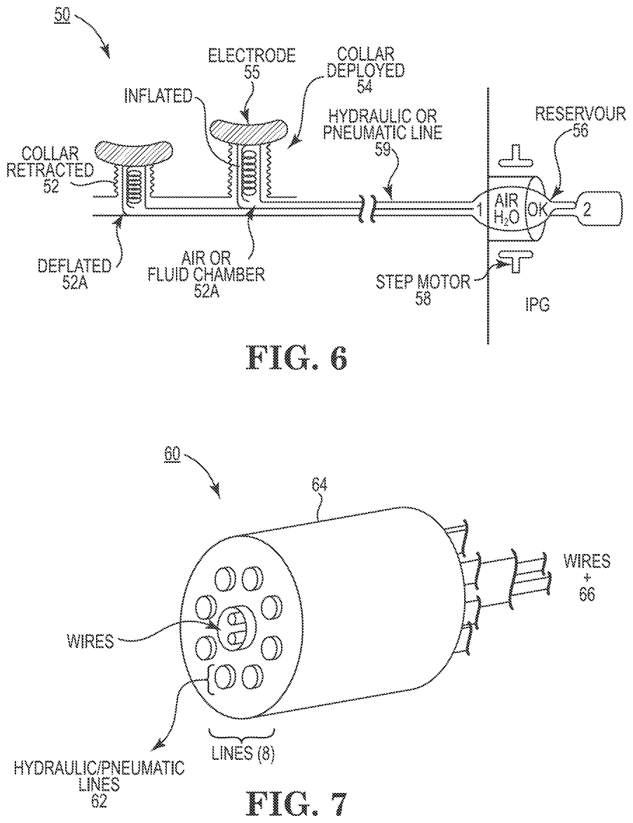

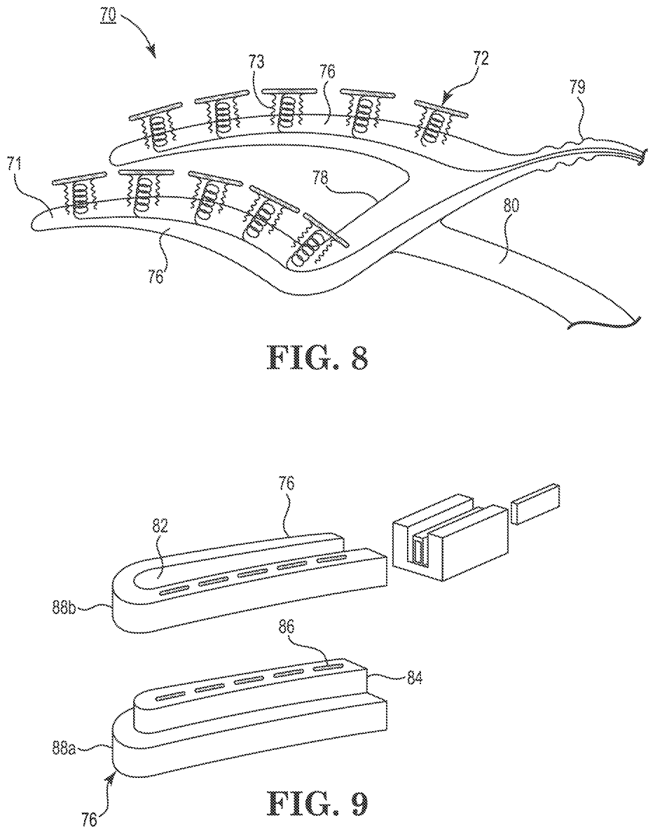

FIG. 8 depicts an open configuration of the embodiment in which neural interface device 70 includes a series of collars 71 and corresponding electrodes 72 are distributed along two parallel arms 76 of the device 70. The device 70 may be a cuff that can be wrapped around the outside of a nerve bundle or NVB in an extravascular embodiment, although it is shown stretched-flat as illustrated in FIG. 8. The arms 76 are joined together by an orthogonal bridge 78 and electrical lines and hydraulic/pneumatic lines are provided by lead 79. An option extension 80 may be used to position the device 70 (e.g. grasping around the NVB). Cuff arms (71, 76) and electrodes 72 may be individually controlled, as any number of collars and electrodes 72 may be activated at a time, thereby changing the size of the neural interface as desired. All of the collars along arms (71, 76) may also be commonly controlled, for example as further illustrated in FIG. 9, in which just portions of the arms 76 are illustrated. Thus, “commonly controlled” electrodes refer to electrodes that are controlled to move synchronously. For example, the electrodes are controlled by a single movement, so that they are controlled by a common action and/or a single pneumatic/hydraulic line and/or a single portion on a collar.

FIG. 9 depicts each arm 76 as having a trench 82 (an elongated narrow opening formed in the arm of the neural interface device 70) in which a collar platform 84 and corresponding pneumatic/hydraulic chamber (not shown) are located. The collar platform 84 may be elastic and enclose the pneumatic/hydraulic chamber. Each collar 84 may include one or more electrodes 86. During a high-pressure condition 88a, pressure is increased in the chamber causing the collar 84 to be deployed and the electrodes contact the target tissue. During a low-pressure condition 88b, pressure is reduced in the chamber causing the elastic collar 84 to collapse or be withdrawn into the trench/trough such that the electrodes are no longer in contact with the target tissue.

In embodiments, an open configuration neural interface device, such as neural interface device 70 of FIG. 8, may incorporate self-adjusting elastic (constant pressure/force) springs capable of free pulsation. The collars 73 of electrodes 72 may be individually mounted on separate elastic (compression) springs, such as elastic spring 39 of FIG. 4C, so that each electrode 72 may individually pulsate with arterial or other environmental pressures. All or some of the collars along arms 71 and 76 may also be commonly mounted on one or more elastic springs. For example, two or more collars may be mounted on a common platform over freely pulsating springs. Trench 82 may be mounted over one or more elastic self-adjusting low-pressure/force metal spring(s), or an alike mechanism, allowing the electrode batch 86 to freely pulsate and self-adjust (in unison) with arterial or other environmental pressures.

Material Design

External coatings may be used on the components of the neural interface device and the leads to prevent foreign body tissue reaction. One example embodiment may be doped with steroid drugs like dexamethasone. Another example embodiment may be coated with Poly-2-hydroyethyl-methacrylate (PHEMA). In embodiments, electrode may be formed of metals having properties including low charge injection and low dissolution. In embodiments, the electrodes may have roughened surfaces for better target tissue contact and electrical properties.

The foregoing description of the examples, including illustrated examples, of the invention has been presented only for the purpose of illustration and description and is not intended to be exhaustive or to limit the invention to the precise forms disclosed. Numerous modifications, adaptations, and uses thereof will be apparent to those skilled in the art without departing from the scope of this invention. The illustrative examples described above are given to introduce the reader to the general subject matter discussed here and are not intended to limit the scope of the disclosed concepts.

What is claimed:

1. A neural interface device including one or more pressure-controlled collars, each of the one or more pressure-controlled collars including one or more electrodes.

2. A neuromodulation system comprising:
a neural interface device according to claim 1;
an implanted pulse generator (IPG); and
a lead body.

3. The neuromodulation system of claim 2, wherein the IPG is configured to deliver electrical pulses to the one or more electrodes and hydraulic or pneumatic pressure to the one or more pressure-controlled collars.

4. The neuromodulation system of claim 2 or 3, wherein the IPG includes at least one liquid or gas reservoir and at least one precision motor configured to apply pressure to the reservoir.

5. The neuromodulation system of any of claims 2-4, wherein the lead body is biocompatible and is configured to conduct electrical currents and pressurized liquid or gas through one or more lines between the IPG and the neural interface device.

6. The neuromodulation system of any of claims 1-5, wherein the lead body is characterized by an undulated configuration and is flexible and stretchable.

7. The neuromodulation system of any of claim 1-3 or 6, wherein the one or more pressure-controlled collars include an elastic pulsatile pneumatic chamber, and wherein each chamber is connected via a line through the lead body to a liquid or gas reservoir.

8. The neuromodulation system of claim 7, wherein the one or more electrodes are mounted on a distal end of the one or more pressure-controlled collars and are configured to contact target tissue when activated by an increase in pressure in the liquid or gas reservoir.

9. The neuromodulation system of either of claim 7 or 8, wherein the increase in pressure is created by a precision motor applying pressure to the liquid or gas reservoir.

10. The neuromodulation system of claim 9, wherein the liquid or gas reservoir and the precision motor are located in the IPG.

11. The neuromodulation system of either of claim 9 or 10, wherein the liquid or gas reservoir and the precision motor are located in a header of the IPG.

12. The neuromodulation system of any of claims 9 to 11, wherein the liquid or gas reservoir and the precision motor are located in an intermediate device between the IPG and the neural interface device.

13. The neuromodulation system of any of claims 1 to 6, wherein each of the one or more pressure-controlled collars is independently controlled.

14. The neuromodulation system of claim 13, wherein each independently controlled collar is connected by a line via the lead body to a liquid or gas reservoir and at least one precision motor configured to apply pressure to the liquid or gas reservoir.

15. The neuromodulation system of any of claim 1 to 3, 5, or 6, wherein two or more of the pressure-controlled collars are commonly controlled.

16. The neuromodulation system of claim 15, wherein each commonly controlled collar is connected by a line via the lead body to a liquid or gas reservoir and at least one precision motor configured to apply pressure to the liquid or gas reservoir.

17. The neuromodulation system of claim 15 of 16, wherein each commonly controlled collar is mounted within a trench formed within the neural interface device, wherein the neural interface device includes a pneumatic or hydraulic chamber connected by a line via the lead body to a liquid or gas reservoir and at least one precision motor configured to apply pressure to the liquid or gas reservoir.

18. The neural interface device of claim 1 or neuromodulation system of claim 2, wherein at least one of the one or more pressure-controlled collars is self-adjusting responsive to external pressure.

19. The neural interface device or neuromodulation system of claim 17, wherein a self-adjusting pressure-controlled collar is mounted on a distal end of a corresponding one of a plurality of elastic springs.

20. The neural interface device or neuromodulation system of claim 18, wherein the one of the plurality of elastic springs is a constant-compression spring or a constant-force spring.

21. The neural interface device or neuromodulation system of any of claims 17 to 20, wherein each of the one or more electrodes are configured to contact target tissue and freely pulsate in response to environmental pressure independent of the other ones of the one or more electrodes.

22. The neural interface device or neuromodulation system of any of claims 17 to 20, wherein each of the one or more electrodes are configured to contact target tissue and freely pulsate in response to environmental pressure in unison with at least one of the other ones of the one or more electrodes.
